(19) United States
(12) Patent Application Publication
(10) Pub. No.: US 2026/0218421 A1
(43) Pub. Date: Jul. 30, 2026

(54) PROCESS FOR PREPARING A MICROFIBER COMPOSED OF MICROALGAE LIVING INSIDE, AND RELATED MICROFIBER

(71) Applicant: DEW S.R.L., PORTO SAN GIORGIO (FM) (IT)

(72) Inventors: Valerio DI GIANNANTONIO, PORTO SAN GIORGIO (FM) (IT); Andrea RINAUDO, MACRA (IT); Federico MALACARNE, TORINO (IT)

(21) Appl. No.: 19/148,587

(22) PCT Filed: Dec. 22, 2023
(86) PCT No.: PCT/IT2023/050294

§ 371 Date: Jul. 16, 2025

(30) Foreign Application Priority Data
Jan. 18, 2023 (IT) I02023000000588

(51) Int. Cl. D01F 1/10; C12N 1/12; D01D 5/06; D01D 5/253; D01F 1/04

(52) U.S. Cl. CPC D01F 1/10; C12N 1/12; D01D 5/06; D01D 5/253; D01F 1/04; D10B 2401/20

(57) ABSTRACT

A process is described for preparing a microfibre with living microalgae inside, including the steps of: preparing and mixing micro-algae and a biomaterial in a nutritional medium; and micro-fluidic spinning of the micro-algae mixture and the biomaterial which is polymerized; wherein the micro-fluidic spinning is carried out in a micro-fluidic spinning chip, provided with a spinning die having a “grooves” section, so as to obtain a grooved section.

The present invention refers to a process for preparing a microfibre containing microalgae living inside it, and to the microfibre thus prepared.

In recent years, the need for an eco-sustainable change and implementations in the field of biotechnology have led to ever-increasing experimentation in the biomaterials sector.

An idea of “Biogarmentry” consisting of a biomaterial with microalgae on its surface is known at an academic level. This idea, however, does not refer to a microfiber with a living organism inside it, as the microalgae are screen-printed on a fabric.

At an industrial level, there are biomaterials made from the fibres of brown algae (e.g. Seacell), which cannot be defined as living, as they are produced from the fibrous part of the algae themselves.

The University of London St. Martins is experimenting, in the master degree in bio-design, with living biomaterials composed of algae.

Scientists at the University of Rochester and Delft University of Technology in the Netherlands have created a living photosynthetic material from algae using 3D printing.

To create the photosynthetic material, the researchers started with non-living bacterial cellulose, an organic compound produced and secreted by bacteria.

A theoretical project is known in the field of design fiction and speculative design; the result of this project is a prototype obtained using common microalgae (for example Hematococcus Pluvialis) and sodium alginate for food use.

This type of sodium alginate (E401) used for spherification in the food sector, from the Special Ingredients company, presents degradation problems after approximately 3÷4 months, compromising the integrity of the microfibre and its contents.

The processed microfibre appears to be living, but with a lifespan of approximately 1 month, which is too short for any use.

Furthermore, a microfibre circular section which causes poor gas exchange capacity.

The object of the present invention is to overcome at least in part the reported drawbacks.

The above and other objects, as will be explained below, are obtained with a production process and the material obtained with the application of the process, respectively as claimed in the related claims.

The production process of a microfibre with living microalgae inside it comprises the following steps:
preparing microalgae and a biomaterial;
mixing the microalgae and the biomaterial in a nutritional medium;
microfluidic spinning.

The microfiber obtained therefore includes a part made by spinning a biomaterial and living microalgae, wherein the microalgae are dispersed within the microfibre.

The microalgae are chosen from those belonging to the aero-terrestrial family, and more generally, microalgae with characteristics of high levels of tolerance to desiccation, or the following living organelles: fungi, cyanobacteria, bioluminescent bacteria, lichens.

The biomaterial is chosen from the following: cellulose, proteins, silk protein, hydrogel, brown algae, collagen, natural rubber, polysaccharides, chitosan, spider silk.

The microfibre thus obtained, with the microalgae inside, is collected on reels.

Preferred embodiments and non-trivial variations of the present invention are the subject matter of the dependent claims.

It is understood that all attached claims form an integral part of this description.

The microfibre with microalgae present inside it, according to the present invention, is living as it is composed of a biomaterial capable of reproducing the ideal survival environment for the microalgae. The microfibre is capable of producing oxygen and filtering CO₂ from the air, as it carries out chlorophyll photosynthesis. It can also respond to changes in the environment by taking on different colours, due to the production of antioxidants.

The microfibre according to the invention is naturally green in colour; the intensity of the colouring can be varied by simply varying the concentration of the microalgae. Furthermore, it is possible to give the microfibre specific colours using different types of pigments, preferably of natural origin.

To perform its function, the microfiber according to the invention requires sunlight and water vapor. In the presence of these elements, the microfibre is able to preserve vitality, which, in optimal conditions, can last up to a period of approximately one year.

The microalgae used have high levels of tolerance to desiccation, preventing senescence through the metabolic block of chlorophyll photosynthesis in environments with scarcity of water and/or sunlight, reactivating the metabolic process in an environment that presents the conditions that microalgae require.

A microfibre is entirely composed of organic materials which make it 100% biodegradable, thus defining a product that can be included in the field of circular economy.

The microfiber according to the invention can be used in the textile field to create a living fabric. This fabric, for the production of clothing, thanks also to its weaving, is able to condense and absorb CO₂ and water vapor from the skin of the person wearing it and from the in surrounding environment. This interaction of the tissue with a human body establishes a symbiotic relationship with the skin, which receives oxygen, proteins, antioxidants and shielding from UV rays; added to these benefits is the fabric characteristic of being fireproof.

In the field of architecture and design, living microfibres can be used to create outdoor and indoor architectural coverings, and more generally to support application uses in the field of green building. Due to the characteristics of the microfibre mentioned above, and the consequent application in architecture, it will be possible to achieve the mitigation of the microclimate in the environment, which consists in the filtering of CO₂ and the production of oxygen.

Finally, given its biodegradability and its very high nutritional intake, living microfibres, at the end of their life cycle, can be disposed of as fertilizer for plants or fodder for farm animals.

It will be immediately obvious that numerous variations and modifications can be made to what is described (for example relating to shape, dimensions, arrangements and parts with equivalent functionality) without departing from the scope of the invention, as appears from the attached claims.

The present invention will be better described by some preferred embodiments thereof, provided by way of example and not by way of limitation, with reference to the attached drawings, in which:

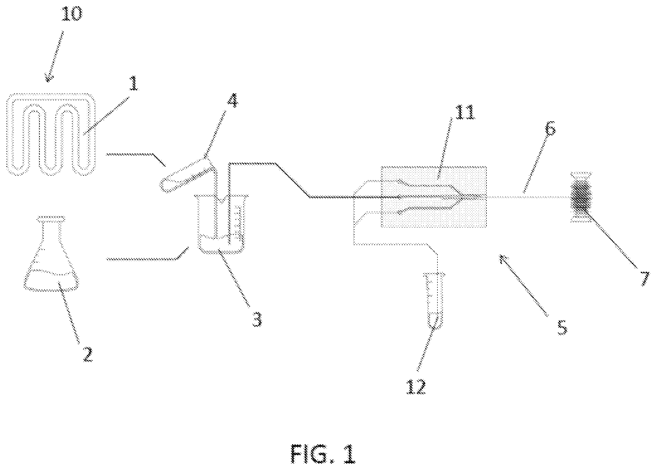

FIG. 1 schematically shows the process for preparing a microfiber with living microalgae, according to the present invention;

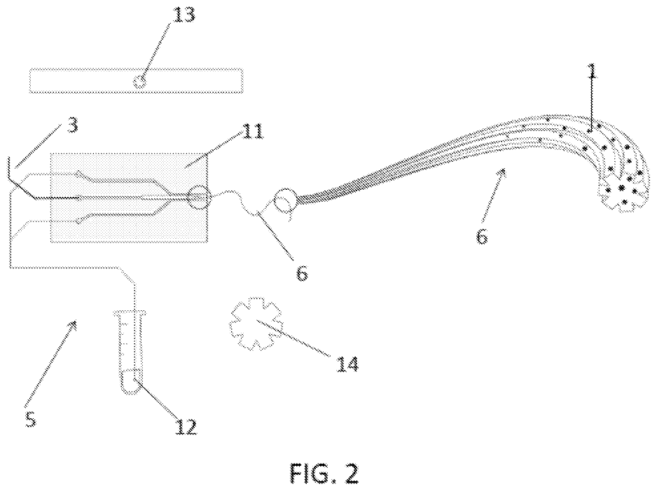

FIG. 2 shows some details of the manufacturing process;

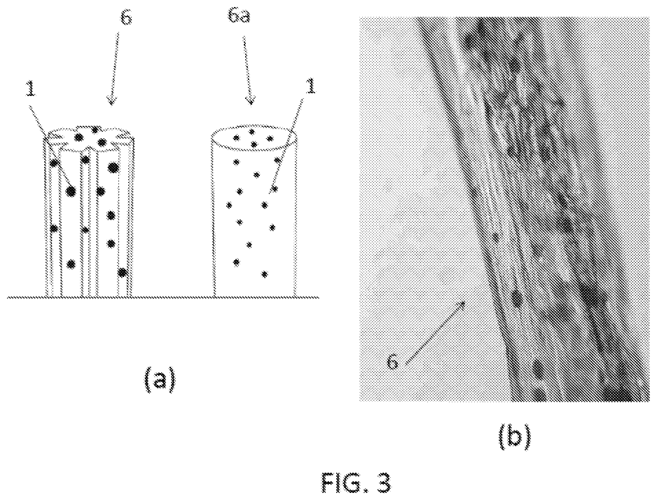

FIG. 3 (a, b) show the comparison between the microfiber according to the invention and the microfiber according to the prior art;

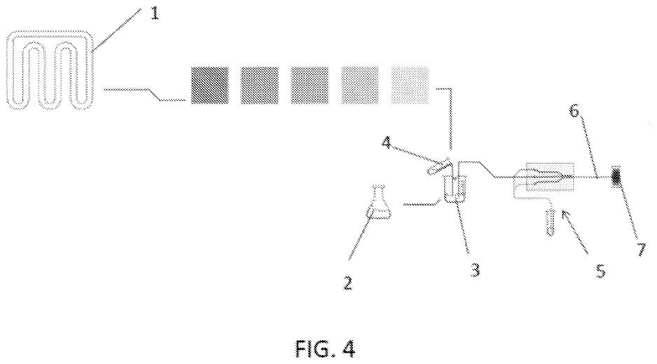

FIG. 4 shows how to obtain microfibers coloured with various shades of green;

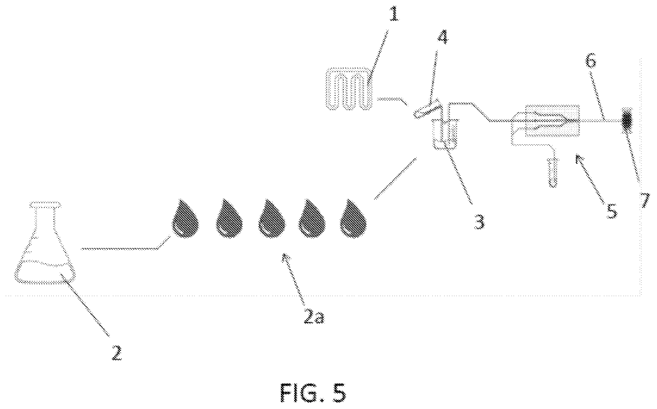

FIG. 5 shows how to obtain microfibers of various colours.

With reference to FIG. 1, the microfiber with living microalgae inside it, according to the invention, includes two main components: microalgae (1) and biomaterial (2).

The production of the microfiber with living microalgae inside it involves the following steps, shown schematically in FIG. 1:
step 1: preparing microalgae (1) and a biomaterial (2);
step 2: mixing (3) the microalgae (1) and the biomaterial (2) in a nutritional medium (4);
step 3: microfluidic spinning (5).

The microfibre (6) thus obtained, with the microalgae (1) inside, is collected on reels (7).

Step 1—The preparation of the microalgae (1) involves culturing them in a bioreactor (10). The bioreactor (10) reproduces, through light and nutrients, a suitable environment for the multiplication and growth of microalgae (1).

According to a preferred embodiment of the invention, the microalgae (1) used have high levels of tolerance to drying, such as those belonging to the family of aero-terrestrial algae, more precisely desert algae (for example Acutodesmus Deserticola). The main characteristic of this microalgae is the high resistance to multiple daily drying cycles, which translates into a better ability to maintain its metabolic composition in a dehydrated environment, and to readjust its metabolism at the time of rehydration. Another characteristic is the ability to obtain a metabolic block of chlorophyll photosynthesis in a dehydrated environment, the block being removable, with the relative restoration of chlorophyll photosynthesis, even after long periods, in the presence of minimal quantities of water. These characteristics allow the microalgae (1) to survive inside a microfibre, even in the absence of rehydration measures for long periods of time, prolonging its vitality.

Among the additional advantages that derive from this use are: greater mechanical resistance and durability of more than 1 year, before seeing the first signs of degradation. Other advantages of this type lie in the high absorbability of the liquids, which guarantees a greater supply of water to the microalgae, and fire-retardant properties.

According to further preferred embodiments of the invention, the following living organelles can be used as an alternative to microalgae: fungi, cyanobacteria, bacteria to survive inside the microfibre (for example bioluminescent bacteria for the production of self-illuminating microfibre).

As regards the biomaterial (2), a type of G and M alginate for textile use is preferably used, with the corresponding additives (for example hydrophilic polymers and graphene nanoparticles) to improve its mechanical characteristics in the polymerization step. This biomaterial is produced from brown algae. Being biocompatible with the human body, it is used in tissue engineering. In this case, it represents the ideal environment for the survival of the microalgae (1) incorporated within it.

The main properties of the biomaterial (2) are biocompatibility with the microalgae (1), biodegradability, flame retardant properties, microbial protection, high absorbability of liquids, gas permeability, polymerization in calcium solution.

Types of alternative biomaterials are the following: cellulose, silk protein, hydrogel, brown algae, collagen, natural rubber, polysaccharides, chitosan, spider silk.

Step 2-Subsequently microalgae (1) and biomaterial (2) are mixed (3) in a nutrient media (4) specific for the chosen microalgae (1) and biomaterial (2), (for example, in the case of microalgae and biomaterial mentioned above, Freshwater BG-11 Medium).

Step 3—The mixture obtained is subjected to microfluidic spinning (5) in a microfluidic spinning chip (11) using the property of alginate to polymerize in calcium solutions (12). The passage of the mixture (3), added with the calcium solution (12) into the spinning microfluidic chip (11), allows the polymerization of the solution (3) of microalgae (1) and biomaterial (2) in the form of microfibre (6) with performance characteristics, in particular due to the fact that the surface for gas exchange has increased compared to the prior art which provided substantially smooth surfaces.

The microchannel inside the chip (11) has a diameter calibrated according to the diameter of the microalgae (1) contained inside, to reduce the distance between the external environment and the microalgae (1) itself, in order to optimize gas exchange.

Using a spinning matrix (13) having a “grooved” section, and with a diameter <0.5 mm, a grooved section (14) of the microfiber (6) is obtained, increasing the surface area for gas exchange compared to the prior art which involved substantially smooth surfaces.

FIG. 3a highlights the difference between the microfibre (6), with a grooved section according to the invention, compared to the microfibre (6a), as obtained according to the prior art. FIG. 3b shows a photographic view of the microfiber (6).

Once polymerised, the microfibre is dried at room temperature for 5-15 min and collected into spools (7).

The biomaterial (2) ensures the survival of the microalgae (1) inside it; due to its gas permeability, it guarantees the gas exchange produced by photosynthesis a nutritional supply for their survival. Its regular rehydration, for example through nebulization, favours the durability of the life of the microfiber over time. Failure to take care of it leads to the metabolic suspension of photosynthesis, but if not exposed to conditions of long drying periods, the microfiber (6) will resume photosynthesis in the presence of water.

In the event that the microfibre (6) is subjected to long periods of lack of hydration and/or nutritional supply, the microalgae (1) will die, whilst maintaining the microfibre biomaterial/bioplastic status unchanged.

As regards colouring, it is possible to produce ranges of different gradients of the green colour (FIG. 4), varying the concentration of microalgae (1) in the solution to be polymerized (3).

Another colouring technique consists of pigmenting the biomaterial (2) (FIG. 5), preferably with natural pigments (2a), before mixing (3) with the microalgae (1). In this way, any type of colour can be obtained.

1. A process for preparing a microfibre (6) with microalgae (1) living inside, characterized in that the process comprises the following steps:
preparing micro-algae (1) and a biomaterial (2);
mixing (3) the microalgae (1) and the biomaterial (2) in a nutritional medium (4);
micro-fluidic spinning (5) of the mixture of micro-algae (1) and the biomaterial (2) which is polymerised;
wherein:
the micro-algae (1) are cultivated in a bioreactor (10), designed to reproduce, through light and nutrients, a suitable environment for the multiplication and growth of the micro-algae (1);
these micro-algae (1) are chosen from those with characteristics of high levels of tolerance to drying;
the micro-algae (1) are chosen from those belonging to the aero-terrestrial family;
the micro-fluidic spinning (5) is carried out in a micro-fluidic spinning chip (11), provided with a spinning matrix (13) having a “grooves” section, so as to obtain a grooved section (14); and
the micro-fluidic spinning (5) is carried out by passing the mixture (3), with the addition of a calcium solution (12), into a micro-fluidic spinning chip (11), the calcium solution (12) causing the polymerization of the mixture (3).

2. The process according to claim 1, characterized in that the biomaterial (2) is chosen from the following: alginate G and M for textile use, cellulose, silk protein, hydrogel, brown algae, collagen, natural rubber, polysaccharides, chitosan, spider silk.

3. The process according to claim 2, characterized in that the biomaterial (2) is added with one or more of the following additives: hydrophilic polymers and graphene nanoparticles, so as to improve its mechanical characteristics in the polymerization step.

4. The process according to claim 1, wherein the micro-algae (1) are replaced by the following living organelles: fungi, cyanobacteria, bioluminescent bacteria.

5. The process according to claim 1, characterized in that the microfibre (6) is given a colour with various green intensities, by varying the concentration of microalgae (1) in the solution to polymerize (3).

6. The process according to claim 1, characterized in that the microfibre (6) is given any colour by adding pigments to the biological material (2).

7. A microfiber (6) comprising a part obtained by spinning a biomaterial (2) and living microalgae (1), characterized in that the microalgae (1) are uniformly dispersed within the microfibre (6).

8. A microfibre (6) comprising a part obtained by spinning a biomaterial (2) and living microalgae (1), characterized in that the microalgae (1) are uniformly dispersed within the microfibre (6), wherein the microfibre (6) is obtained according to a process according to claim 1.
